(19) United States
(12) Patent Application Publication
(10) Pub. No.: US 2011/0306664 A1
(43) Pub. Date: Dec. 15, 2011

(54) MEDICAL DIETARY COMPOSITION FOR TREATING NEURODEGENERATIVE DISEASES

(75) Inventors: Anna PETRONI, Segrate (MI) (IT); Sebastiano BANNI, Cagliari (CA) (IT); Marco CAPPA, Roma (RM) (IT)

(73) Assignee: SOFEDUS S.R.L., Milano MI (IT)

(21) Appl. No.: 13/159,681

(22) Filed: Jun. 14, 2011

Related U.S. Application Data
(63) Continuation-in-part of application No. PCT/EP2009/067070, filed on Dec. 14, 2009.

(30) Foreign Application Priority Data
Dec. 15, 2008 (IT) MI2008A002221

(51) Int. Cl. A61K 31/231; A61P 25/28

(52) U.S. Cl. 514/547

(57) ABSTRACT

Medical dietary composition for treating neurodegenerative diseases such as Alzheimer, Parkinson's disease, and more preferably adrenoleukodystrophy comprising a mixture of glyceryl trioleate (GTO), glyceryl trierucate (GTE) and at least one conjugated linoleic acid as active ingredient, in combination with suitable excipients and/or diluents.

### CROSS-REFERENCE TO RELATED APPLICATIONS

This application is a Continuation-in-Part of PCT/EP2009/067070 filed on Dec. 14, 2009 which claims priority to and the benefit of Italian Application No. MI2008A002221 filed on Dec. 15, 2008, the contents of which are incorporated herein by reference.

### FIELD OF THE INVENTION

The present invention relates to a medical dietary composition for treating neurodegenerative diseases and in particular for treating adrenoleukodystrophy.

### PRIOR ART

Adrenoleukodystrophy (X-ALD, OMIM #300100) is a progressive neurodegenerative disease associated to the low oxidation of non-branched long chain saturated fatty acids (VLCFA; Very Long-Chain Fatty Acid) (Igarashi M., Schaumburg H. H., Powers J., Kishimoto Y., Kolodny E., Suzuki K., 1976. Fatty acid abnormality in adrenoleukodystrophy. Journal of Neurochemistry 26, 851-860), particularly hexacosanoic (C26:0) and tetracosanoic (C24:0) acid. Accumulation of VLCFA as cholesteryl esters in the plasma, adrenal cortex and white substance of the brain is due to a defect of the beta oxidation of the fatty acids at the peroxisomal level (Singh I., Moser A. E., Moser H. W., Kishimoto Y., 1984. Adrenoleukodystrophy: impaired oxidation of very long chain fatty acids in white blood cells, cultured skin fibroblasts and amniocytes. Pediatric Research 18, 286-290.). The most clear clinical manifestations are a progressive neurodegeneration process, demyelination, and hypogonadism as well as alopecia in some phenotypes. The defective gene is in the disease map on gene Xq28 and encodes a peroxisomal transport protein ABCD1 (ATP-binding cassette) belonging to subfamily D, member 1, renamed ALDP. The disease is characterised by the presence of proteins homologous to ALDP belonging to the same family of membrane transporters on peroxisomes ALDR, PMP70, and P70R respectively encoded by genes ABCD2, ABCD3 and ABCD4.

As of date, the treatment available for patients suffering from ALD is a therapy based on Lorenzo's Oil, a 4:1 mixture of glyceryl trioleate (oleic acid, 18:1) and glyceryl trierucate (erucic acid, 22:1) which, combined with a reduction of the saturated fatty acids in the diet, seems to normalize the levels of VLCFA (Rizzo W. B., Leshner R. T., Odone A., Dammann A., Craft D. A., 1989. Dietary erucic acid therapy for X-Adrenoleukodystrophy. Neurology, 39, 1415-1422) in patients.

The problem associated to this type of therapy lies in the fact that one of the constituents, erucic acid, hardly passes through the blood-brain barrier (Uptake and metabolism of plasma-derived erucic acid by rat brain, 2006. Golovko and Murphy E. J. J. Lipid Res. 47, 1289-1297) and therefore it has a low effect on the nervous system. Furthermore, clinical studies wherein Lorenzo's Oil has been used showed a reduction of the VLCFAs but not an improvement in the neurodegenerative processes (Follow up of 89 asymptomatic patients with adrenoleukodystrophy treated with Lorenzo's Oil, 2005. Moser H. W., Raymond G. V. et al. Arch. Neurol. 62:1073-1080).

Conjugated linoleic acids (CLA) are C18 fatty acids containing two ethylenic unsaturations which, contrary to linoleic acid (18:2, n-6), are conjugated.

The most common naturally-occurring CLAs hitherto isolated are those respectively having double bonds in positions 6-8, 7-9, 8-10, 9-11, 10-12, 11-13, 12-14. Each of these acids may be in form of 4 geometric isomers and cis/cis isomer, trans/trans isomer, cis/trans isomer and trans/cis isomer respectively.

Therefore, 24 naturally occurring CLAs have been identified up to now.

Of these, the most abundant are the 9cis-11 trans-CLA and 10trans-12cis-CLA isomers.

The best source of CLA in nature is food of animal origin especially ruminant meat and milk as well as dairy products thereof. However, CLA may also be found in eggs and fish fat as well as sea animals. Low concentrations of CLA are also found in vegetable oils.

The best source of CLAs are dairy products containing more than 75% of 9cis-11trans, and 9trans-11 cis isomers.

This type of acids has several therapeutic functions, among the main ones are an anticarcinogenic activity (see Banni S., Angioni E, Carta G, Mc. Ginley J, Thompson HJ, Barbano D: “Conjugated linoleic acid enriched butter alters mammary gland morphogenesis and reduces cancer risks in rats”. J., Nutr. (129) 2135-2142 (1999) mainly due to the abovementioned 9cis-11 trans-CLA (see Lavillonieres F., Chajes V., Martin, J. C. Sebedio, J. L. Lhuillery, C. Bougnoux P., Dietary purified cis9, transl l-conjugated linoleic acid isomer has anticarcinogenic properties in chemically induced mammary tumors in rats. Nutrition and Cancer 2003 45 190-194,). 10trans-12cis CLA isomer has a considerable impact on the metabolism of fats and thus it is used in weight loss diets in that it reduces abdominal fat (see for example: Thom, E, Wadstein J, Gudmunsen O. September-October 2001 “Conjugated linoleic acid reduces body fat in healthy exercising humans” The Journal of Medical Research 29(5) 392-396), and for treating diabetes in that it reduces the absorption of glucose (see for example J. W. Ryder, C. P. Portocarro, X. M. Song, L. Cui, M. Yu, T. Combatsiaris, D. Galuska, D. and Barman, D. E. Baumn. D. M. Barbano, Mj Charron, J. R. Zierath and K. L. Houseknecht “Isomer Specific Antidiabetic Properties of Conjugated Linoleic acid” Diabetes (50)1149-1157 2001).

Experiments on animal models showed how CLA is capable of passing through the blood-brain barrier in that it is incorporated in the brain. “Incorporation and metabolism of c9-t11 and t 10-c12 conjugated linoleic acid (CLA) isomer in rat brain.” Mauro Fa, Andrea Diana, Lina Cordeddu, Maria Paola Melis, Elisabetta Murru, Valeria Sogos, Sebastiano Banni. Biochimica et Biophysica Acta 1736 (2005) 61-66.

### SUMMARY OF THE INVENTION

The Applicant has now discovered that the association of glycerol trioleate, glycerol trierucate with at least one conjugated linoleic acid is particularly effective in treating neurodegenerative diseases and adrenoleukodystrophy in particular.

As a matter of fact, as observed in the experimental part of the present description, alongside having a positive impact due to the reduction of VLCFA 26:0, the association of the abovementioned active ingredients lies in the fact that—contrary to erucic acid—the CLA promptly passes through the blood-brain barrier in subjects suffering from neurodegenerative diseases and in particular the above-mentioned adrenoleukodystrophy, thus having a positive effect also at a neurophysiological level.

In fact from the clinical trials described herein below an improvement in Somatosensory Evoked Potential was observed in ALD patients treated with the compositions of the invention, whereas this effect was absent in patients treated with Lorenzo's oil. Thus, medical dietary compositions containing GTO, GTE as active ingredient and at least one conjugated linoleic acid in combination with suitable diluents and/or excipients are an object of the present invention.

### DESCRIPTION OF THE FIGURES

FIG. 1 discloses Plasma levels of 26:0 at different time points (* indicates p<0.05 versus t0).

FIG. 2 discloses Plasma 22:6/22:0 ratio at different time points (* indicates p<0.05 versus t0).

FIG. 3 describes: CLA and 22:6/20:5 levels in plasma and CSF at different time points (* indicates p<0.05 versus t0).

FIG. 4 reports Plasma levels of the two components of LO (oleic and erucic acids) at different time points (* indicates p<0.05 versus t0).

### DETAILED DESCRIPTION OF THE INVENTION

According to the present invention, the term “medical dietary composition” means a composition belonging to the category of “dietary foods for special medical purposes” which are a category of products for particular nutrition, regulated by the 89/398 EEC Community Directive, ratified in Italy by the Legislative Decree 111/92. In other words, these are foods formulated based on reliable principles of medicine and nutritional science to effectively meet the particular nutritional needs of the subjects they are intended for according to generally accepted scientific data (Guidarelli, Copparoni and Scarpa: “Prodotti destinati ad una alimentazione particolare” Renzo Editore, pages 17-21, II° Edition).

The weight ratio of glyceryl trioleate (oleic acid, 18:1) and glyceryl trierucate (erucic acid, 22:1) is preferably 4:1.

The GTO+GTE mixture and the conjugated linoleic acid are preferably present in the medical dietary composition according to the present invention at volumetric ratio comprised between 1:20 and 20:1. More preferably the GTO+GTE mixture and CLA are present in the dietary composition of the invention at weight ratio respectively comprised between 3:1 and 15:1. Even more preferably between 7:1 and 11:1. The presence of CLA in the composition of the invention has a further advantage, since it reduces the daily intake of erucic acid. In fact erucic acid is known to cause in the long run myocardial lipidosis (“Effects of Dietary Saturated Fat on Erucic Acid Induced Myocardial lipidosis in Rats” J. K. G. Kramer, F. D. Sauer, Miss. Wolynetz E. R. Farnworth and K. M. Johnston, Lipids vol. 27 no. 8). The total amount of said active ingredients is preferably comprised between 1 and 100% in weight on the total weight of the medical dietary composition.

As used herein, the term conjugated linoleic acid is used to indicate any linoleic acid having ethylenic unsaturations at any position from C2-C4 to C15-C17 hence 14 isomers. Given that each of these isomers may have 4 geometric isomers i.e. cis/trans, trans/cis, cis/cis, trans/trans, the composition according to the present invention may contain at least one out of the 56 spatial isomers of the conjugated linoleic acid.

At least one conjugated linoleic acid having the ethylenic unsaturations in the positions comprised between C7-C9 to C12-C14 is preferably used for the objects of the present invention. Thus, the compositions subject of the present invention preferably contain at least one out of the 24 isomers wherein the ethylenic unsaturations are respectively present in C7-C9, C8-C10, C9-C11, C10-C12, C11-C13, C12-C14.

More preferably the conjugated acids subject of the present invention are the geometric isomers of the conjugate having the ethylenic unsaturations respectively at carbon 9 and at carbon 11 and the one with the ethylenic unsaturations at carbon 10 and at carbon 12 and their mixtures. Thus, the compositions subject of the present invention more preferably contain at least one of the eight geometric isomers having the ethylenic unsaturations at positions C9-C11, and C10-C12

Even more preferably they are selected from among cis/trans and trans/cis isomers of said conjugated acids and according to the most preferred embodiment they are a mixture of 9cis-11 trans CLA and 10trans-12cis CLA. Said mixture is present at weight ratios preferably comprised between 1:20 and 20:1, The medical dietary compositions according to the present invention are in oral, liquid oil or soft capsules form.

Assessment tests regarding the efficiency of the compositions subject of the present invention, provided for exemplifying and non-limiting purposes, are provided below.

### Mixture Effectiveness Test

### Mixture Preparation

50 ml of a CLA mixture—c9,t11 and t10,c12 in 1:1 ratio—are added to 500 ml containing as the main component a of a GTO/GTE 4:1 mixture.

The formulation in the form of an oil thus obtained has therefore the following final composition expressed as percentage by weight

61.3% oleic acid,
15.0% erucic acid,
9.9% conjugated linoleic acid.
In the above mixture therefore the weight ratio of Lorenzo's oil main component (GTO+GTE) with respect to CLA is about 8 (7.7)

### Experiment

5 female patients affected by X-ALD, heterozygote, is recruited. The following Table 1 summarizes the clinical characteristics and the results of molecular analysis of the five patients. 50 ml per day of the oil mixture prepared as above described corresponding therefore respectively to a Lorenzo's oil (LO) dosage of 40 g/day and conjugated linoleic acid (CLA) 5 g/day were administered to these patients for two months.

The following Table 1 summarizes the clinical characteristics and the results of molecular analysis of the five patients.

TABLE 1

| | age | molecular | aminoacid | clinical |
|---|---|---|---|---|
| patient | (years) | defect | substitution | symptoms |
| 1 | 58.99 | c.994C > T | p.Gln332x | spastic paraparesis, |
| | | | | adrenal |
| | | | | insufficiency |
| 2 | 50.24 | c.560P > L | p.Pro560Leu | depression |
| 3 | 44.59 | c.1885 | p.Asp629Asn | NO |
| 4 | 49.96 | c.1027G > A | p.Gly343Ser | peripheral |
| | | | | neuropathy, |
| | | | | depression |
| 5 | 51.22 | C.1252C > T | p.Arg418Trp | NO |

After recruitment, during the 1st visit (t0), blood samples were collected from all the patients in order to analyze their baseline platelet count (Zinkham W H, Kickler T, Borel J., Moser H W. Lorenzo's Oil and Thrombocytopenia in Patients with Adrenoleukodystrophy N Engl J Med 1993; 328:1126-1127) and plasma lipid profile (including the fatty acid profile). At baseline, the somatosensory evoked potentials (SEPs) of upper and lower limbs were also performed in all the patients. The 2nd visit (t1) was scheduled after 4 weeks of treatment with LO+CLA to repeat blood sampling for platelet count and lipid analyses.

The 3rd visit (t2) was scheduled after 4 more weeks (8 weeks from baseline), to perform the second evaluation of the SEPs, and to repeat blood sampling for platelet count and lipid analyses.

One of the symptomatic patients with spastic paraparesis (patient 1) had been previously treated with LO and had stopped LO therapy one month before recruitment.

At baseline and during the 3rd visit, aliquots of cerebrospinal fluid (CSF) were collected by lumbar puncture in all patients. Blood and CSF samples were centrifuged and stored at −80° C., until analyses.

The CSF samples were analysed for fatty acid composition, in order to verify whether CLA and erucic acid are able to pass, and to what extent, the blood-brain barrier.

The institutional review board approved the study, written informed consent was obtained from all patients at recruitment.

### Lipid Analyses

Total lipids were extracted from plasma or CSF using chloroform/methanol 2:1 (v/v) (Folch J, Lees M, Sloane-Stanley GH. A simple method for the isolation and purification of total lipid from animal tissues. J. Biol. Chem. 1957; 226: 497-509). Separation of phospholipids from total lipids was performed as previously reported (Banni S, Carta G, Angioni E, Murru E, Scanu P, Melis MP, et al. Distribution of conjugated linoleic acid and metabolites in different lipid fractions in the rat liver. J Lipid Res 2001; 42: 1056-1061.). Aliquots were mildly saponified as previously described (Banni S, Carta G, Contini Angioni E, Deiana M, Dessi M A, et al. Characterization of Conjugated Diene Fatty Acids in Milk, Dairy Products, and Lamb Tissues. J Nutr Biochem 1996; 7: 150-155), in order to obtain free fatty acids for HPLC analysis. Separation of fatty acids was carried out by an Agilent 1100 HPLC system (Agilent, Palo Alto, Calif., USA) equipped with a diode array detector as previously reported (Angioni E, Lercker G, Frega N G, Carta G, Melis M P, Murru E, et al. UV spectral properties of lipids as a tool for their identification. Eur J Lipid Sci Technol 2002; 104: 59-64).

Since saturated fatty acids are transparent to UV, they were measured, after methylation, by means of a gas chromatograph (Agilent, Model 6890, Palo Alto, Calif.) equipped with split ratio of 20:1 injection port, a flame ionization detector (FID), an autosampler (Agilent, Model 7673), a 100 m HP-88 fused capillary column (Agilent), and an Agilent ChemStation software system. The injector and detector temperatures were set at 250° C. and 280° C. respectively. H2 served as carrier gas (1 ml/min), and the FID gases were H2 (30 ml/min), N2 (30 ml/min), and purified air (300 ml/min). The temperature program was as follows: initial temperature was 120° C., programmed at 10° C./min to 210° C. and 5° C./min to 230° C., then programmed at 25° C./min to 250° C. and held for 2 min.

A further identification of fatty acids, and in particular of CLA and its metabolites has been carried out by LC-APCI-MS as previously described (Banni et al., 2004).

### Cytokine Analyses

CSF samples were centrifuged at 1500 rpm for 10 min and the supernatants were immediately frozen at −80° C. IL-6 and IL-8 levels were quantified in CSF using sandwich immunoassays. All reagents were purchased from R&D System (R&D System Inc., Minneapolis, Minn., USA). IL-6 was quantified using the Quantikine® High Sensitivity human IL-6 Immunoassay (Catalog Number: HS600B) according to the manufacturer's instructions. The detection range of the kit was 0.15-10 pg/ml.

IL-8 was quantified using a combination of a monoclonal anti-human CXCL8/IL-8 capture antibody (Catalog number: MAB208), a byotinilated human CXCL8 detection antibody (Catalog number: BAF208) and recombinant human CXCL8 (Catalog Number: 208-IL) as the standard. The detection range of this assay was 7.0-2000 pg/ml.

### Somatosensory Evoked Potential (SEP) Recording Procedure

SEPs were recorded to both median and tibial nerve stimulation before and after 8 weeks' treatment with mixture of LO (40 g/day) with CLA (5 g/day).

For SEP recording we used the same procedure described in a previous study (Restuccia D, Di Lazzaro V, Valeriani M, Oliviero A, Le Pera D, Colosimo C, et al. Neurophysiological abnormalities in adrenoleukodystrophy carriers. Evidence of different degrees of central nervous system involvement. Brain 1997; 120 (Pt 7): 1139-48.

Subjects lay on a bed in a quiet and semi-darkened room. Stimuli (0.3 ms duration, 5 Hz) were delivered by skin electrodes at the wrist for the median nerve, and at the ankle for the tibial nerve. Stimulus intensity was adjusted to slightly above the motor threshold. The stimulation rate was 1.5 Hz. An automatic artefact-rejection system excluded from the average all runs containing transients exceeding ±65 mV at any recording channel. In order to ensure baseline stabilization, SEPs were digitally filtered off-line by means of a digital filter with a bandpass of 30-3000 Hz. Two averages of 1000 trials each were obtained for each stimulated nerve. For median nerve SEP recording, electrodes were placed in the supraclavicular fossa (Erb's point), over the sixth cervical vertebra (CV6), and in the parietal scalp regions contralateral and ipsilateral to the stimulated side. Erb's point electrode was referred to an electrode placed in the frontal region (Fz, according to the 10-20 Intenational System), the CV6 electrode was referred to an electrode located immediately above the thyroid cartilage (AC), and the scalp electrodes were referred to the shoulder contralateral to the stimulated side (noncephalic reference). For tibial nerve SEP recording, electrodes were placed over the fourth lumbar (L4) vertebra [referred to the second lumbar (L2) vertebra], over the twelfth dorsal (T12) vertebra (referred to an electrode located immediately above the umbilicus—Abd), at the Cz location of the 10-20 International System (referred to Fz) and in the temporal region (T3/T4) contralateral to the stimulation (referred to an electrode in the ipsilateral parietal region).

For the median nerve SEPs, we evaluated the peak latencies of: 1) the peripheral N9 potential, generated by the brachial plexus volley and recorded by the Erb's electrode, 2) the spinal N13 response, generated within the cervical grey matter and identifiable in the CV6-AC trace, 3) and 4) the scalp far-field P9 and P14 responses, recorded from the scalp electrodes and generated in the brachial plexus and in the lower brainstem respectively, and 5) the contralateral N20 and P25 responses, recorded from the parietal electrode and generated by neurons in the primary somatosensory (SI) area. We also calculated the P9-N20 interpeak interval to assess the conduction time in central somatosensory pathways, and the P9-P14 and P14-N20 interpeak intervals, to evaluate the somatosensory conduction along the cervical dorsal columns and the intracranial lemniscal pathways respectively. Lastly, we evaluated the peak-to peak amplitudes of the spinal N13 response, the P14 far-field scalp potential and the N20 scalp response (Desmedt J E, Cheron G., 1980 Central somatosensory conduction in man: neural generators and interpeak latencies of far-field components recorded from neck and right or left scalp and earlobes. Electroencephalogr Clin Neurophysiol 1980; 50:382-403; Yamada T, Ishida T, Kudo Y, Rodnitzky R L, Kimura J. Clinical correlates of abnormal P14 in median SEPs. Neurology 1986; 36:765-771.). For the tibial nerve SEPs, we evaluated the peak latencies of: 1) the cauda equina response (CE), recorded at L4 and generated by the peripheral volley along the L5-SI roots, 2) the spinal N24 response, identifiable in the T12-Abd trace and generated by the lumbo-sacral dorsal horn neurons, 3) and 4) the scalp N37 and P40 responses, recorded from the contralateral temporal electrode and the Cz lead respectively and probably generated in the SI area. Moreover, the peak-to-peak amplitudes of the N24 potential and of both the scalp N37 and P40 SEP components were measured. Finally, we calculated the N24-P40 interpeak interval to assess the conduction time in central somatosensory pathways.

The SEP values obtained in our patients were referred to the normative data for upper and lower limb SEPs reported in our previous studies (Restuccia et al., 1993; Restuccia et al., 1994; Valeriani et al., 2000)

### Statistic Analysis

SEP latencies and amplitudes were compared between both sides and between before and after treatment. Paired Student's t-test was used to compare the SEP latencies and the interpeak intervals, while Wilcoxon-test was used to compare the SEP amplitudes. Statistic significance was fixed at p<0.05.

Wilcoxon-test was used to compare lipid profile findings at different time points.

Due to the small number and the wide scatter of the data, cytokine analysis results are showed as raw data without statistic analysis.

### Results

### Clinical Data

No significant adverse effects, potentially related to the administration of the mixture of LO+CLA, were observed during the study period. One patient presented with severe headache after the lumbar puncture, that requested hospitalization for two days.

In 3 out of 5 subjects, neurological examination was normal before and after treatment. One patient had a spastic paraparesis (patient 1) and another patient showed a mild tactile and thermic sensory loss in lower limbs (patient 4) before treatment. Their clinical examination did not change after treatment.

### Lipid Metabolism Findings

The results of lipid analyses and platelet count are reported in the following Table 2.

TABLE 2
Plasma lipid profile and platelet count at the different time points

| | | | | total | | | LDL | | | HDL | | | | | |
|---|---|---|---|---|---|---|---|---|---|---|---|---|---|---|---|
| | triglycerides | | | cholesterol | | | cholesterol | | | cholesterol | | | Platelet count | | |
| | (mg/dl) | | | (mg/dl) | | | (mg/dl) | | | (mg/dl) | | | ×10⁹/L | | |
| patient | t0 | t1 | t2 | t0 | t1 | t2 | t0 | t1 | t2 | t0 | t1 | t2 | t0 | t1 | t2 |
| 1 | 61 | 66 | 104 | 264 | 261 | 250 | 141 | 156 | 134 | 87 | 92 | 95 | 346 | 312 | 303 |
| 2 | 93 | 120 | 108 | 239 | 250 | 219 | 162 | 163 | 136 | 58 | 63 | 61 | 164 | 205 | 246 |
| 3 | 48 | 39 | 50 | 179 | 178 | 191 | 92 | 96 | 102 | 77 | 74 | 79 | 357 | 324 | 283 |
| 4 | 43 | 54 | 67 | 179 | 175 | 165 | 119 | 101 | 91 | 51 | 53 | 61 | 264 | 209 | 218 |
| 5 | 84 | 83 | 61 | 195 | 190 | 149 | 120 | 122 | 106 | 59 | 51 | 41 | 249 | 223 | 286 |

LO+CLA did not significantly affect platelet count. Total cholesterol was reduced during therapy in 4 out of 5 individuals, LDL decreased while HDL tended to increase, and triglycerides were relatively unchanged (table 2). The changes in cholesterolemia could be ascribed to the particularly high intake of oleic acid, which is widely known to decrease LDL cholesterol (Hu et al., 1997).

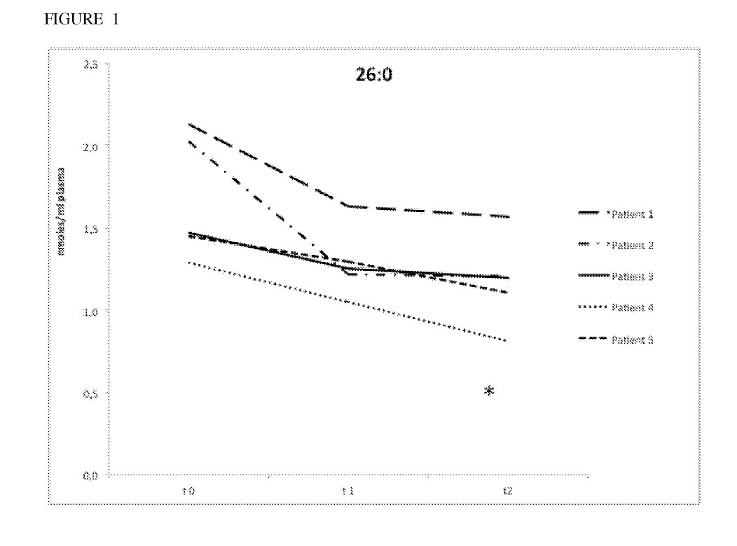

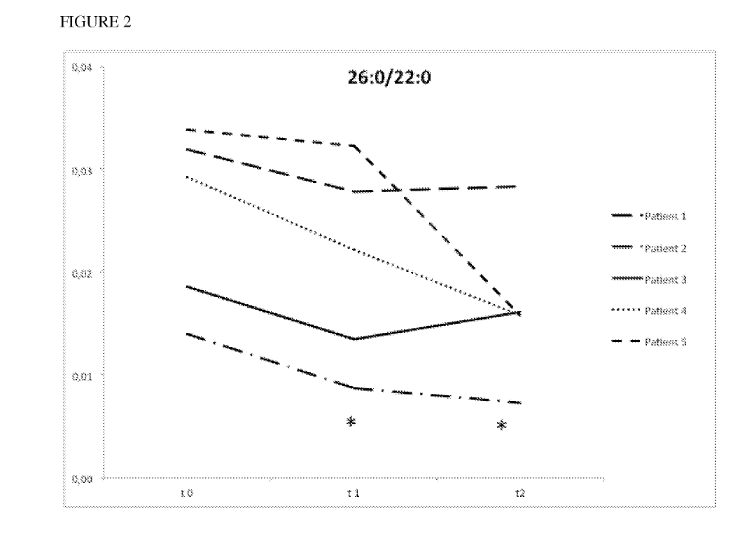

FIG. 1 clearly shows that the mixture LO+CLA treatment decreases plasma levels of 26:0 in all patients. This is also true for the 22:6/22:0 ratio (FIG. 2).

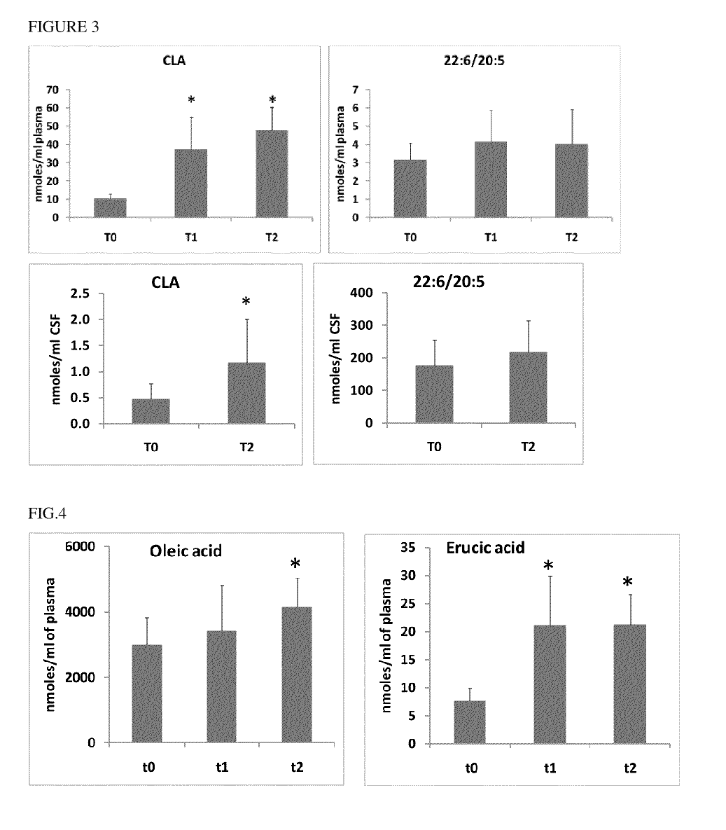

Treatment with the mixture LO+CLA significantly increases CLA levels in plasma (FIG. 3A) and in CSF (FIG. 3C). The 22:6/20:5 ratio, an indirect marker of peroxisomal beta oxidation, since to form 22:6 from 20:5 a step in peroxisome is obligatory (Ferdinandusse et al., 2001), also increases in plasma and CSF but not significantly (FIGS. 3B and D, respectively).

FIG. 4 depicts the increase of plasma levels of the 2 components of LO. Both oleic and erucic acids significantly increase, even though the rise of oleic acid becomes significant only after two months. No changes were detected in CSF (data not shown).

### Cytokine Assessment

IL-8 was not detectable in the CSF (data not shown). On the contrary, at study entry IL-6 was clearly detectable in CSF of all patients. After LO+CLA administration, IL-6 levels were decreased in 3 out of 5 patients (patients 1-3), while remained substantially unchanged in 2 patients (patients 4 and 5). The results are shown in table 3.

TABLE 3
IL-6 levels in CSF at different time points

| | | IL-6 |
|---|---|---|
| | | (pg/ml) |
| Patient 1 | t0 | 5.62 |
| | t2 | 1.63 |
| Patient 2 | t0 | 1.71 |
| | t2 | <0.15 |
| Patient 3 | t0 | 3.74 |
| | t2 | 2.88 |
| Patient 4 | t0 | 2.04 |
| | t2 | 1.96 |
| Patient 5 | t0 | 2.65 |
| | t2 | 2.88 |

### SEP

Table 4 summarizes SEP results.

TABLE 4
Summary of the results of the somatosensory evoked potentials evaluation of the upper
and lower limbs at baseline and after 8 weeks

| | Median nerve | | | | | | | | | |
|---|---|---|---|---|---|---|---|---|---|---|
| | N9lat | | N13lat | | P14 lat | | N20lat | | N9-N20 | |
| | t0 | t2 | t0 | t2 | t0 | t2 | t0 | t2 | t0 | t2 |
| Mean | 9.84 | 9.78 | 13.28 | 13.29 | 14.87 | 14.98 | 20.45 | 20.35 | 10.61 | 10.57 |
| SD | 0.38 | 0.41 | 0.42 | 0.53 | 0.65 | 0.56 | 1.16 | 1.19 | 0.92 | 1.02 |
| p | 0.639 | | 0.931 | | 0.329 | | 0.501 | | 0.828 | |

| | | | |
|---|---|---|---|
| N9 amp | N13 amp | P14 amp | N20 amp |

| | t0 | t2 | t0 | t2 | t0 | t2 | t0 | t2 |
|---|---|---|---|---|---|---|---|---|
| Mean | 2.44 | 3.06 | 0.39 | 0.40 | 0.44 | 0.38 | 0.38 | 0.36 |
| SD | 0.66 | 1.36 | 0.16 | 0.17 | 0.15 | 0.11 | 0.17 | 0.12 |
| p | 0.061 | | 0.901 | | 0.150 | | 0.608 | |

| | Tibial nerve | | | | | | | |
|---|---|---|---|---|---|---|---|---|
| | N24 | | P40 | | N37 | | N24-P40 | |
| | latency | | latency | | latency | | interval | |
| | t0 | t2 | t0 | t2 | t0 | t2 | t0 | t2 |
| Mean | 22.75 | 22.04 | 47.16 | 40.83 | 45.33 | 40.49 | 24.40 | 18.79 |
| SD | 1.82 | 1.20 | 4.59 | 4.47 | 4.03 | 4.06 | 5.59 | 3.67 |
| p | 0.282 | | 0.003 | | 0.019 | | 0.016 | |

| | | |
|---|---|---|
| N24 | P40 | N37 |
| amplitude | amplitude | amplitude |

| | t0 | t2 | t0 | t2 | t0 | t2 |
|---|---|---|---|---|---|---|
| Mean | 0.42 | 0.61 | 0.17 | 0.66 | 0.269 | 0.396 |
| SD | 0.25 | 0.18 | 0.12 | 0.82 | 0.1963 | 0.1699 |
| p | 0.1 | | 0.05 | | 0.08 | |

### Neurophysiologic Findings Before Treatment

SEP amplitudes and latencies were not different between right and left side stimulation (p>0.05). Median nerve SEPs were normal in all patients. After tibial nerve stimulation, the CE response and spinal N24 potential were within normal limits in all patients. The scalp P40 potentials resulted abnormally delayed bilaterally in 4 out of 5 subjects (patients 1-4). The central somatosensory conduction after both the right and left tibial nerve stimulation (N24-P40 interval) was prolonged in 4 out of 5 patients (patients 1-4).

Neurophysiologic Findings after Treatment

SEP amplitudes and latencies were not different between right and left side stimulation (p>0.05).

Median nerve SEPs remained normal in all patients and did not differ from those recorded before treatment (p>0.05). After tibial nerve stimulation, the scalp P40 potential resulted abnormally delayed only in 1 out of 5 subjects (patient 1). Conversely, in 3 other patients showing a delayed P40 latency before treatment (patients 2, 3 and 4), the P40 potential was recorded with a normal latency after treatment. After treatment, the average P40 latency in our patients decreased significantly from 47.16±4.59 ms to 40.83±4.47 ms (p: 0.003) and the N37 latency decreased from 45.33±4.03 ms to 40.49±4.05 ms (p: 0.019). Moreover, the N24-P40 interpeak interval decreased from 24.40±5.59 ms to 18.79±3.67 ms (p: 0.016). Lastly, treatment led to a slight increase of the both N37 and the P40 amplitudes. Overall, in 4 out of 5 patients (patients 2-5) tibial nerve SEPs were significantly improved after treatment.

### CONCLUSIONS

Our study demonstrates for the first time the prompt entry of CLA through the blood-brain barrier in humans. The additional therapeutic effect of LO+CLA is that LO inhibits VLCFA accumulation, mainly in peripheral tissues. In addition, CLA increases the remaining activity of ALDP, in peripheral tissues and brain, as confirmed by the increased 22:6/20:5 ratio both in plasma and in CSF.

The treatment with LO+CLA results in an improvement of the SEPs. A better SEP is a sign of neurological improvement which implies an ongoing physiological recovery. The evident decrease of IL-6 concentrations in CSF, in 3 out of 5 patients after LO+CLA administration, could represent the biochemical support of this finding. In the present study, 4 out 5 patients showed a prolonged central somatosensory conduction time to lower limb stimulation before treatment, confirming a previous report in ALD carriers (Restuccia D, Di Lazzaro V, Valeriani M, Oliviero A, Le Pera D, Colosimo C, et al. Neurophysiological abnormalities in adrenoleukodystrophy carriers. Evidence of different degrees of central nervous system involvement. Brain 1997; 120 (Pt 7): 1139-48.). In our study, tibial nerve SEPs fully recovered after treatment in 3 out 4 patients who showed abnormal tibial nerve SEPs at baseline. This result is particularly important, when compared with the findings of a previous study performed in adult ALD patients, in which no SEP improvement was obtained by dietary LO supplementation (Restuccia D, Di Lazzaro V, Valeriani M, Oliviero A, Le Pera D, Barba C, et al. Neurophysiologic follow-up of long-term dietary treatment in adult-onset adrenoleukodystrophy. Neurology 1999; 52: 810-6.).

In conclusion, the synergistic action of LO+CLA can revert the progressive somato-sensory conduction impairment in ALD carriers. The results of this pilot study are encouraging for planning a larger multi-center trial, that could contribute to open the field for new therapeutic strategies, not only for ALD, but also for other neurodegenerative diseases where inflammation plays a central role.

1. A medical dietary composition comprising as the active ingredient an association of glyceryl trioleate (GTO), glyceryl trierucate (GTE) and an association of at least one conjugated linoleic acid as the active ingredient in combination with suitable excipients and/or diluents.

2. The composition according to claim 1, wherein the GTO and the GTE is present at weight ratios of 4:1 with respect to each other.

3. The composition according to claim 1, wherein the GTO plus GTE and the conjugated linoleic acid are present in volumetric ratios respectively in the range between 1:20 and 20:1.

4. The composition according to claim 1, wherein the GTO plus GTE and the conjugated linoleic acid are present in weight ratio respectively comprised between 3:1 and 15:1.

5. The compositions according to claim 4, wherein said weight ratio is comprised between 7:1 and 11:1.

6. The compositions according to claim 1, wherein the total concentration of the active ingredient is in the range between 1 and 100% by weight on the total weight of the composition.

7. The composition according claims 1, comprising at least one conjugated linoleic acid selected among the 24 isomers having the ethylenic unsaturation at C7-C9, C8-C10, C9-C11, C10-C12, C11-C13, C12-C14.

8. The composition according to claim 1, wherein the conjugated linoleic acid comprises a mixture of 9-cis-11-trans conjugated linoleic acid and 10 trans-12-cis conjugated linoleic acid.

9. The composition according to claim 8, wherein the weight ratio between 9 cis-11 trans conjugated linoleic acid and 10 trans-12 cis is in the range between 1:20 and 20:1.

10. The composition according to claim 1, for treating neurodegenerative diseases.

11. The composition according to claim 10, for treating adrenoleukodystrophy.

12. A therapeutic method for the treatment of adrenoleukodystrophy comprising administering a subject in need of such a treatment a therapeutically effective amount of an association of glyceryl trioleate (GTO), glyceryl trierucate (GTE), and conjugated linoleic acid, wherein:
a) the GTO and GTE are present in weight ratio of respectively 4:1;
b) the GTO+GTE and conjugated linoleic acid are in weight ratio respectively comprised between 7:1 and 11:1.

13. The therapeutic method according to claim 12, wherein said GTO+GTE and said conjugated linoleioc acid are administered in the form of a single dietary medical composition.
